(19) United States
(12) Patent Application Publication
(10) Pub. No.: US 2020/0178878 A1
(43) Pub. Date: Jun. 11, 2020

(54) METHOD FOR SWALLOW SAFETY MONITORING AND DEVICE THEREOF

(71) Applicant: National Applied Research Laboratories, Taipei City (TW)

(72) Inventors: HUNG-MIN SUN, Taipei City (TW); TZU-I TSENG, Taipei City (TW); SHU-HUI HUNG, Taipei City (TW)

(73) Assignee: National Applied Research Laboratories, Taipei City (TW)

(21) Appl. No.: 16/670,186

(22) Filed: Oct. 31, 2019

(30) Foreign Application Priority Data
Dec. 11, 2018 (TW) 107144616

(51) Int. Cl. A61B 5/00; A61B 5/08; A61B 7/00

(52) U.S. Cl. CPC A61B 5/4205; A61B 5/082; A61B 2562/0219; A61B 7/003; A61B 2562/0204; A61B 5/0823

(57) ABSTRACT

The present invention provides a method for swallow safety monitoring and a device thereof, which can monitor eating safety in real-time. By means of the real-time records of fundamental breath sound, the feeding safety for patients or weakness persons with dysphagia can be real-timely monitored and alarmed.

### FIELD OF THE INVENTION

The present invention relates generally to a method for monitoring and a device thereof, and particularly to a method for swallow safety monitoring and a device thereof.

### BACKGROUND OF THE INVENTION

The tube feeding diet is to inject fluid food into a nasogastric tube, a nasoduodenal tube, or a nasojejunum tube, or through esophagostomy, gastrostomy, or jejunostomy with the purpose of providing appropriate nutrition support for patients or weakness persons with dysphagia or those with sufficient gastrointestinal functions but unable to diet through mouths. In general, the tube feeding diet uses a nasogastric tube (N-G tube) for feeding. By definition, an N-G tube is a tube going from the nose to the stomach.

The material of the tube is most polyethylene or silicone with softness and slight elasticity. Depending on the number, the diameters of tubes differ. The greater the number is, the larger the diameter of tube will be. For general adults, the tube number are 12 Fr to 16 Fr. There is a black mark on the tube for recording its reaching depth.

In the process of tube feeding, because the vertical distance between the syringe and the bottom of a patient's stomach is about 45 cm, the feeding rate must be controlled. In general, to feed 200 to 400 ml of food, the feeding time should be within 15 to 25 minutes. In other words, the feeding rate is around 13 to 16 ml per minute. During the process, if the fed patient appears abnormal, the feeding process must be paused immediately and the medical staff must be notified. The abnormal conditions include:
1. Sudden severe cough or tachypnea
2. Large pushing resistance and unable to feed
3. Abdominal pain or vomit, not improving after pause feeding
4. N-G tube sliding out, or not sure if the N-G tube still in the stomach
5. The color of withdrawn residue appearing brown, dark red, or florid (need to exclude the color of food)

Generally, caregivers are hired in homecare or care centers to care receivers such as the elderly or patients. Unfortunately, the care quality of current caregivers differs or a caregiver needs to care multiple care receivers. Many care receivers suffer from dysphagia owing to aging-induced muscle relaxation, diseases, or surgeries. If the dysphagia is not improved effectively under assistance, physiologically, malnutrition might occur while mentally, depression will happen due to inability in enjoying food. Accordingly, the qualities of work and life of patients or weakness persons with dysphagia and caregivers can be enhanced if technologies are applied to tube feeding.

### SUMMARY

An objective of the present invention is to provide a method for swallow safety monitoring and a device thereof, which provide assistive monitoring for relieving long-term care loads of patients' family or caregivers, as well as preventing infection caused by food backflow and reducing death caused by complications.

To achieve the above objective, the present invention discloses a method for swallow safety monitoring, comprising steps of: disposing one or more sound sensor at one or more chest location of a patient; sensing a breath sound at the chest location of the patient for generating a reference sound datum; sensing a plurality of fundamental breath sensing sounds at the chest location of the patient in a diet period for generating a plurality of sound sensing data; and comparing the plurality of sound sensing data according to the reference sound datum and monitoring during the diet and swallowing period. Thereby, a user can compare the plurality of sound sensing data with the reference sound datum. By judging if the fundamental breath sensing sounds are normal, the diet condition of the patient can be determined, and hence relieving long-term care loads of patients' family or caregivers as well as preventing infection caused by food backflow and reducing death caused by complications.

According to an embodiment of the present invention, the step of sensing a breath sound at the chest location further comprises sensing a first horizontal angle for generating a reference angle datum; the step of sensing a plurality of fundamental breath sensing sounds at the chest location in a diet period further comprises sensing a second horizontal angle for generating a plurality of sensing angle data. In addition, the method according to the present embodiment further compares the plurality of sensing angle data according to the reference angle datum for monitoring the safety during the whole diet period.

According to an embodiment of the present invention, the method further comprises a step of transmitting the reference sound datum, the plurality of sound sensing data, and the whole diet period to an electronic device.

According to an embodiment of the present invention, the step of comparing the plurality of sound sensing data according to the reference sound datum and monitoring during the whole diet period further comprises steps of comparing the plurality of sound sensing data by using the reference sound datum as a threshold value; and generating an alarm message when the frequency or amplitude of one of the plurality of sound sensing data exceeds the threshold value.

According to another embodiment of the present invention, a device for swallow safety monitoring is disclosed, in which the device comprises one or more sound sensor, a timer, and a monitoring unit. The sound sensor is disposed at one or more chest location for sensing a breath sound and generating a reference sound datum. The timer is connected to the sound sensor and synchronous with the sound sensor for triggering the sound sensor to sense a plurality of sensing sounds and generate a plurality of sound sensing data during a diet period. The monitoring unit is connected to the sound sensor and the timer and monitors according to the reference sound datum, the plurality of sound sensing data, and the diet period.

According to another embodiment of the present invention, the device for swallow safety monitoring further comprises an angle sensor and a storage unit. The angle sensor senses the horizontal positions before and during the diet period for generating a reference angle datum and a plurality of sensing angle data, respectively. The monitoring unit further monitors the postural angles of a patient according to the reference angle datum and the plurality of angle sensing data. Besides, the storage unit stores the reference sound datum, the plurality of sound sensing data, the reference angle datum, the plurality of angle sensing data, and the whole diet period.

According to another embodiment of the present invention, the device for swallow safety monitoring further comprises a transmission module, which is connected to the storage unit for transmitting a first audio datum, a second audio datum, and the diet period to an electronic device via a wireless transmission interface.

### BRIEF DESCRIPTION OF THE DRAWINGS

FIG. 1 shows a flowchart according an embodiment of the present invention;

FIG. 2 shows a schematic diagram of a device according to an embodiment of the present invention;

FIG. 3 shows a partial flowchart according to an embodiment of the present invention;

FIG. 4 shows a schematic diagram of a device according to another embodiment of the present invention;

FIG. 5 shows a schematic diagram of a device according to another embodiment of the present invention;

FIG. 6 shows a flowchart according to another embodiment of the present invention;

FIG. 7 shows a schematic diagram of a device according to another embodiment of the present invention;

FIG. 8 shows a partial flowchart according to another embodiment of the present invention; and

FIG. 9 shows a schematic diagram of a device according to another embodiment of the present invention.

### DETAILED DESCRIPTION

In order to make the structure and characteristics as well as the effectiveness of the present invention to be further understood and recognized, the detailed description of the present invention is provided as follows along with embodiments and accompanying figures. The concepts of the present invention can be embodied by various forms. Those embodiments are not used to limit the scope and range of the present invention.

The present invention provides a method for swallow safety monitoring and a device thereof. By means of the fundamental breath sounds, whether swallowing in a diet is normal can be monitored. Once an abnormal condition occurs, an alarm is submitted to caregivers for intervention.

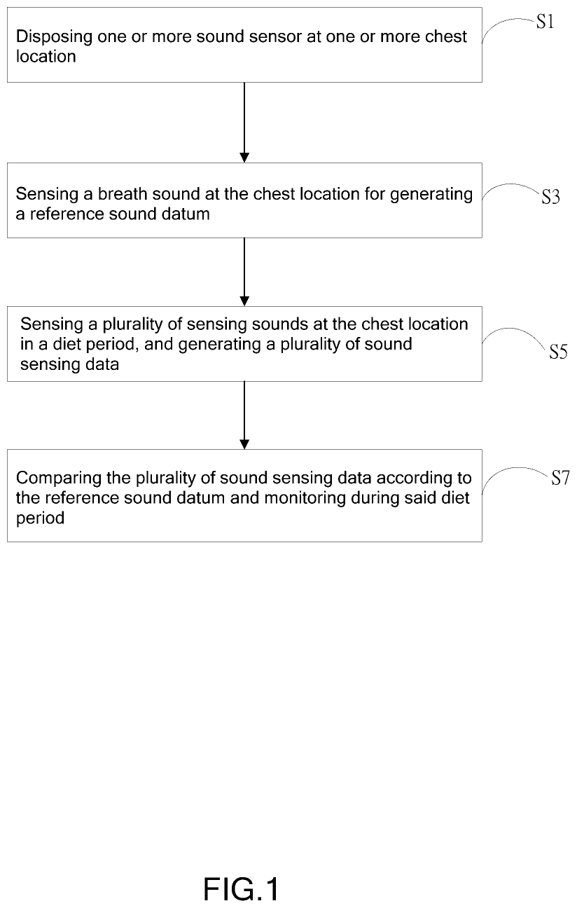

First, please refer to FIG. 1, which shows a flowchart according to an embodiment of the present invention. As shown in the figure, the method for swallow safety monitoring is applied for tube feeding, the method comprises steps of:
Step S1: Disposing one or more sound sensor at one or more chest location;
Step S3: Sensing a breath sound at the chest location for generating a reference sound datum;
Step S5: Sensing a plurality of sensing sounds at the chest location in a diet period, and generating a plurality of sound sensing data; and
Step S7: Comparing the plurality of sound sensing data according to the reference sound datum and monitoring during said diet period.

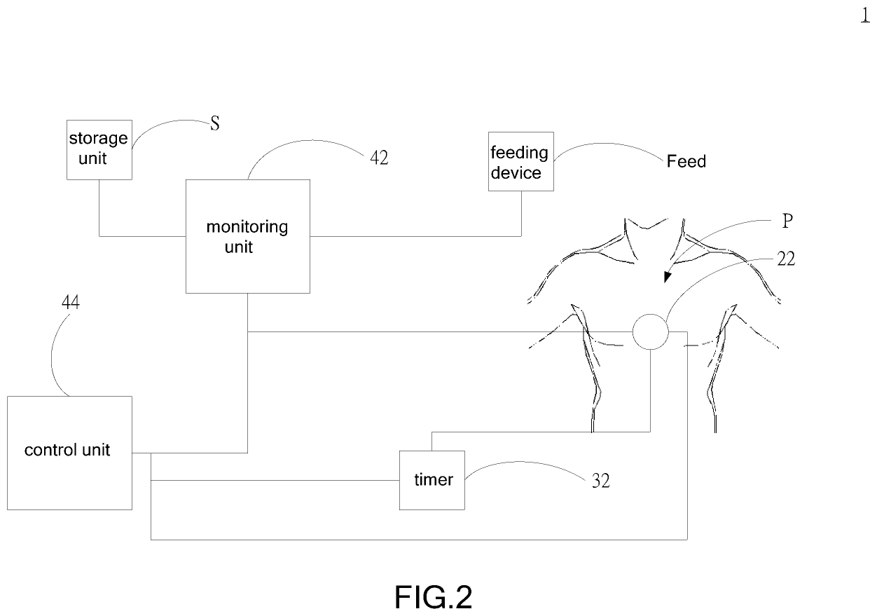

Next, a device for swallow safety monitoring, that is, a device for achieving the method for swallow safety monitoring according to the present invention will be described. Please refer to FIG. 2, which shows a schematic diagram of a device according to an embodiment of the present invention. As shown in FIG. 2, the device for swallow safety monitoring 1 according to the present invention comprises one or more sound sensor 22, a timer 32, and a monitoring unit 42. In addition, the device for swallow safety monitoring 1 according to the present invention can further comprises a control unit 44 and a storage unit S. The sound sensor 22 is disposed at a chest location P. The timer 32 is connected to the sound sensor 22 and thereby synchronous with the sound sensor 22. The monitoring unit 42 is connected to the sound sensor 22 and the timer 32 and monitors the swallowing condition of a care receiver. Furthermore, the control unit 44 is connected to the monitoring unit 42, the timer 32, and the sound sensor 22, and the monitoring unit 42 is further connected with a feeding device Feed.

As shown in the step S1, according to the present embodiment, the sound sensor 22 is disposed at a single chest location P. Nonetheless, the present invention is not limited to the location. A plurality of sound sensors 22 can be disposed around the chest location P for increasing sensing accuracy, for example, at the breastbone, which is a flatter location on the chest. As shown in the step S3, the sound sensor 22 senses the fundamental breath sound at the chest location P in a non-diet period and hence giving a relatively normal breath sound as the reference sound. Then the sound sensor 22 can generate a reference sound datum, which is used as the monitoring standard for the monitoring unit 42. As shown in the step S5, the control unit 44 controls the timer 32 to start timing and controls the sound sensor 22 to start sensing. The timer 32 times in the whole diet period, which includes the time before, within, and after the diet period.

Thereby, the sound sensor 22 senses sound at the chest location P, for example, the bottom edge of the chest, in the whole diet period and generates a plurality of sound sensing data for further comparing by the monitoring unit 42.

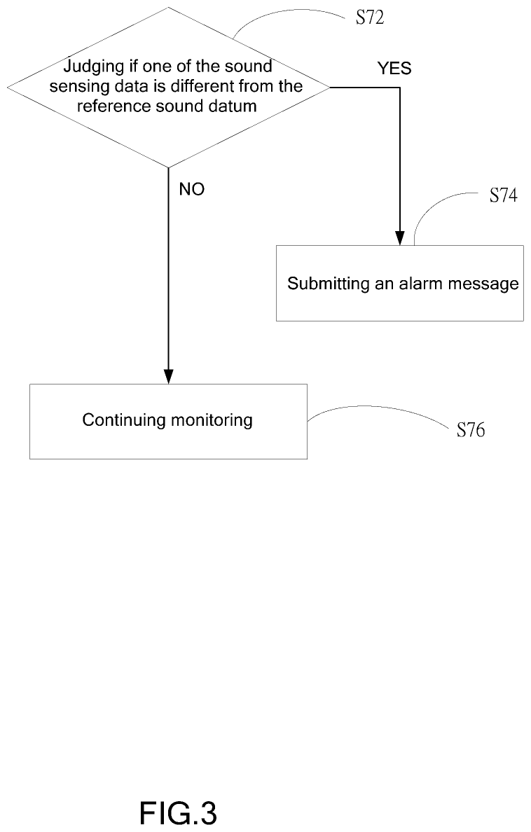

As shown in the step S7, the monitoring unit 42 compares the received reference sound datum with the sound sensing data. According to the comparison result, the swallow sound can be monitored. Please further refer to FIG. 3, which shows a partial flowchart according to an embodiment of the present invention. As shown in the figure, the step S7 further comprises steps of:
Step S72: Judging if one of the sound sensing data is different from the reference sound datum;
Step S74: Submitting an alarm message; and
Step S76: Continuing monitoring.

As shown in the step S72, the monitoring unit 42 compares each of the sound sensing data. By comparing each of the sound sensing data with the reference sound datum, whether each of the sound sensing data is abnormal can be judged. In other words, the reference sound datum is used as a threshold value. When the frequency or amplitude of one of said plurality of sound sensing data exceeds the threshold value, the step S74 will be executed to trigger the monitoring unit 42 to generate an alarm message. When there is no abnormal condition, namely, when the frequency or amplitude of one of said plurality of sound sensing data does not exceed the threshold value, the monitoring unit 42 continues monitoring and returns to the step S5. The storage unit S stores the reference sound datum, the plurality of sound sensing data, and the diet period.

The threshold value as described above can further be the standard value of personal swallow practice in rehabilitation. Extract the sound of swallow practice and compare it with the threshold value. When the frequency or amplitude of one of said plurality of sound sensing data exceeds the threshold value, the step S74 will be executed to trigger the monitoring unit 42 to generate an alarm message. When there is no abnormal condition, namely, when the frequency or amplitude of one of said plurality of sound sensing data does not exceed the threshold value, the monitoring unit 42 continues monitoring and returns to the step S5.

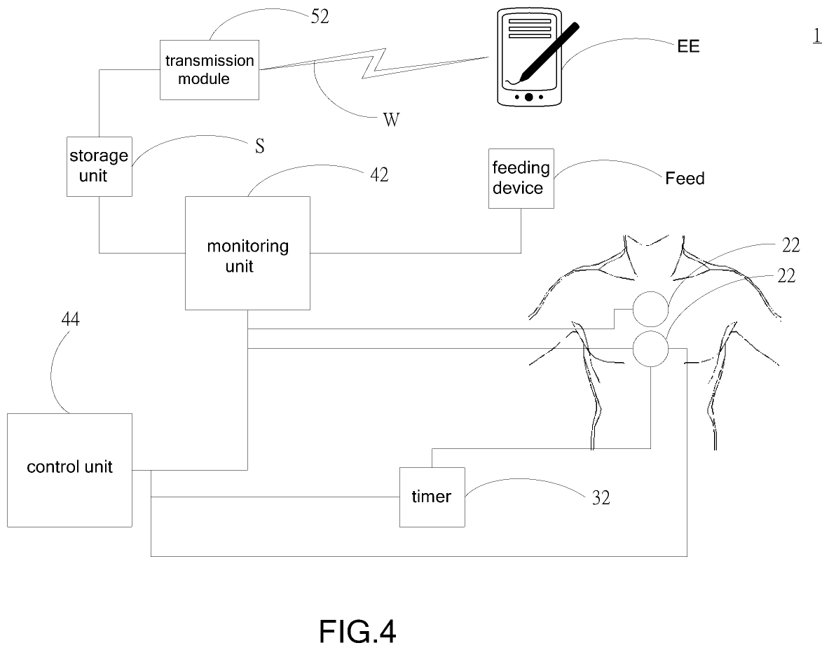

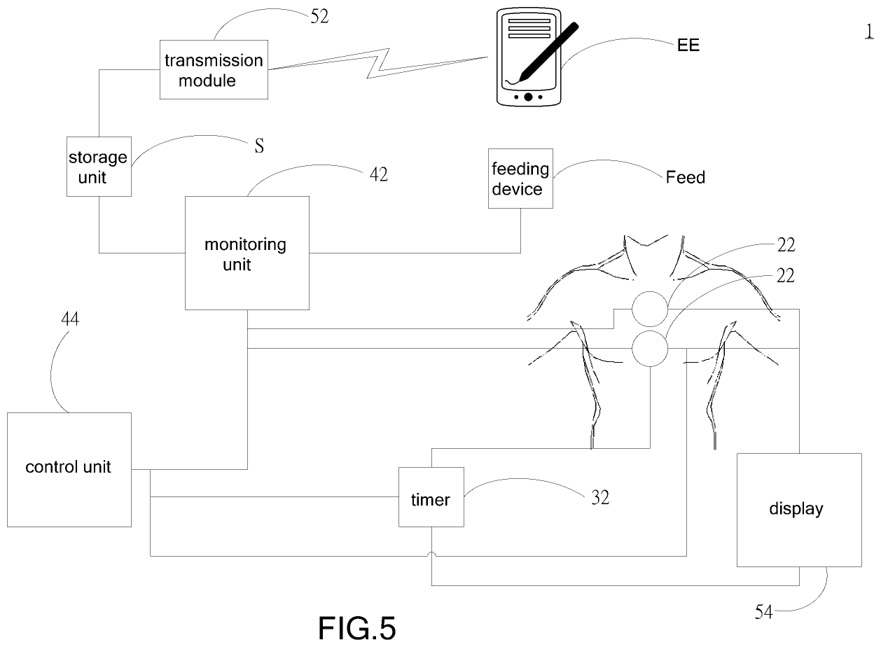

Moreover, as shown in FIG. 4, the device for swallow safety monitoring 1 according to the present invention can further comprises a transmission module 52 connected to the storage unit S for transmitting the reference sound datum, the plurality of sound sensing data, and the diet period to an electronic device EE. Given the fact that everyone owns a smartphone, people can know the diet condition of patients from the electronic device EE promptly. In addition, as shown in FIG. 5, the device for swallow safety monitoring 1 according to the present invention can further comprises a display 54 connected to the sound sensors 22 and the timer 24 for displaying the corresponding data of the sound sensors 22 and the timer 24. For example, the reference sound datum, the plurality of sound sensing data, and the timing information of the timer 24 can be displayed.

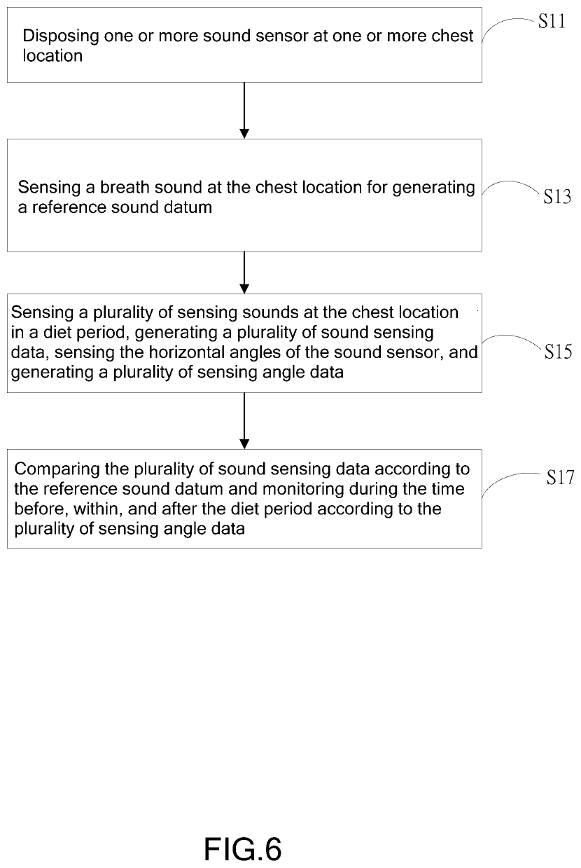

In the following, a flow chart of a method for swallow safety monitoring according to another embodiment of the present invention will be described. Please refer to FIG. 6. The process according to the present invention comprises steps of:
Step S11: Disposing one or more sound sensor at one or more chest location;
Step S13: Sensing a breath sound at the chest location for generating a reference sound datum;
Step S15: Sensing a plurality of sensing sounds at the chest location in a diet period, generating a plurality of sound sensing data, sensing the horizontal angles of the sound sensor, and generating a plurality of sensing angle data; and
Step S17: Comparing the plurality of sound sensing data according to the reference sound datum and monitoring during the time before, within, and after the diet period according to the plurality of sensing angle data.

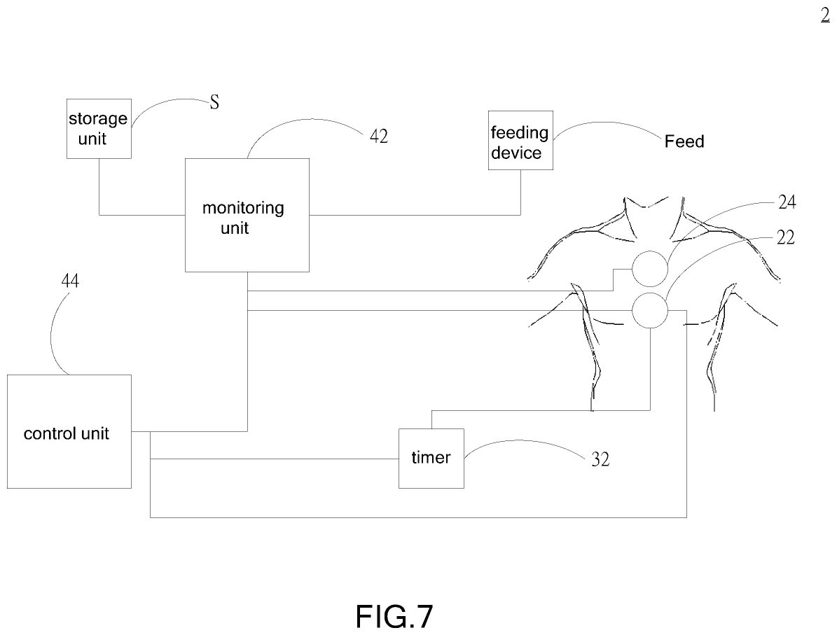

Please refer to FIG. 7. The difference between FIG. 2 and FIG. 7 is that an angle sensor 24 is further disposed at the chest location P and connected to the monitoring unit 42.

In the steps S11 and S13, the execution methods are identical to the technology described in the step S1 above. Hence, the details will not be described again. In the step S15, in addition to monitoring the whole diet period, the sound sensor 22 is further adopted to sense the sound at the chest location P as well as using the angle sensor 24 to sense the body angle of the care receiver. Thereby, a plurality of sound sensing data and a plurality of angle sensing data are generated. For example, attaching the angle sensor 24 to the care receiver's shoulder, waist, or bed for sensing the body angle.

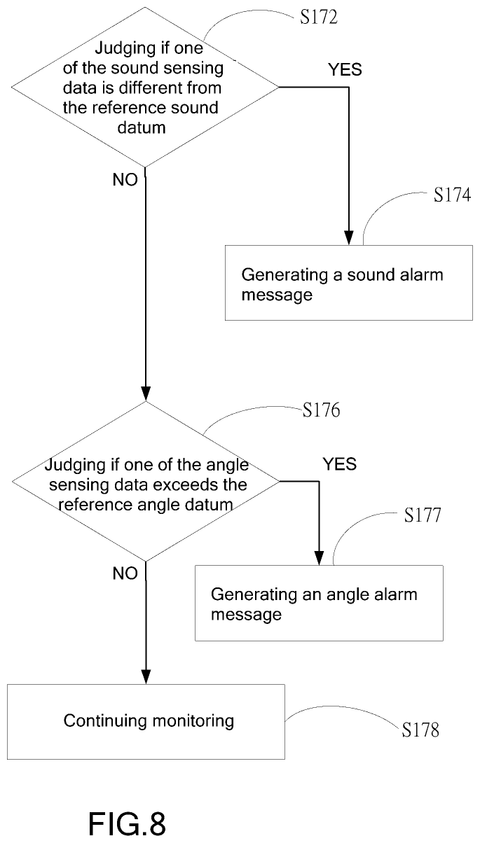

Please refer to FIG. 8. Compared to FIG. 3, the step S17 according to the method for swallow safety monitoring of the present invention further comprises:
Step S172: Judging if one of the sound sensing data is different from the reference sound datum;
Step S174: Generating a sound alarm message;
Step S176: Judging if one of the angle sensing data exceeds the reference angle datum;
Step S177: Generating an angle alarm message;
Step S178: Continuing monitoring.

The step S172 adopts the same technology as the step S72. Hence, the details will not be described. In the step S172, when one of the sound sensing data exceeds the reference sound datum, the step S174 is executed; when one of the sound sensing data does not exceed the reference sound datum, the step S176 is executed. In the step S174, the monitoring unit 42 will generate a sound alarm message as the sound is abnormal. In the step S176, when the monitoring unit 42 judges that one of the angle sensing data exceeds the reference angle datum, the step S177 is executed; when the monitoring unit 42 judges that one of the angle sensing data does not exceed the reference angle datum, the step S178 is executed. Hence, the process returns to the step S15. In the step S177, the monitoring unit 42 will generate an angle alarm message as the angle is abnormal.

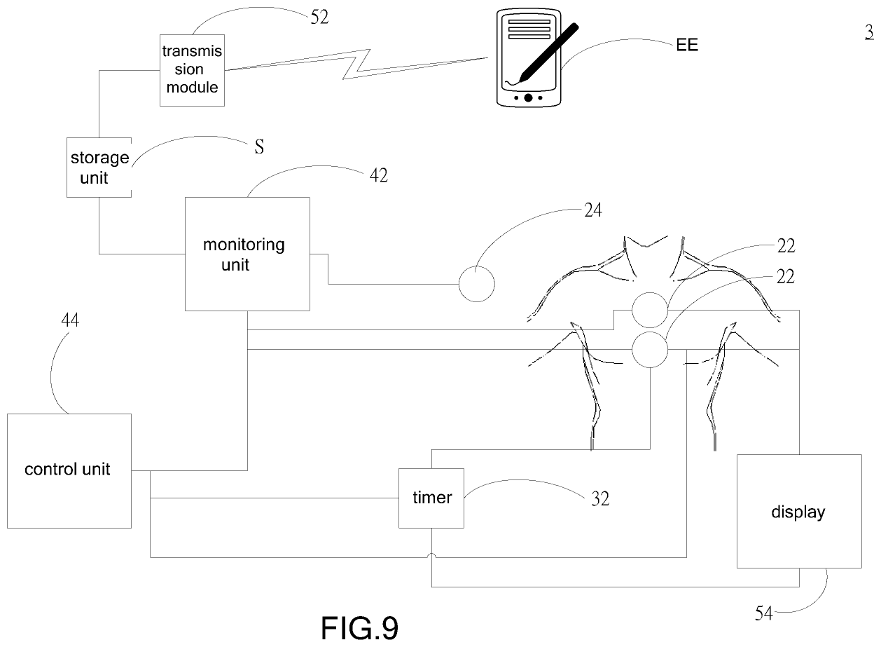

As shown in FIG. 9, the device for swallow safety monitoring 3 directly monitors non-tube-feeding people with dysphagia. Thereby, their swallowing conditions are monitored in a diet period for avoiding danger, such as dyspnea or food impaction caused by choking of food in the throat. Likewise, the device for swallow safety monitoring 1 monitors non-tube-feeding people by means of the steps S11 through S17. The sound sensor 22 acquires the reference sound datum. The monitoring unit 42 judges sensing sound according to the acquired reference sound datum. Meanwhile, the angle sensor 24 senses the body angle or bed angle of people for monitoring the swallowing of people. Besides, the present invention can be applied to home swallow rehabilitation of people.

To sum up, the present invention provides a method for swallow safety monitoring and a device thereof. By using monitoring unit 42 as an automatic assistive monitoring device, the following effects can be achieved:
1. Providing safety service by monitoring breath data before, within, and after a diet through the AI technology;
2. Providing personal precise medical and care mechanism;
3. Relieving long-term care loads of patients' family or caregivers;
4. Preventing infection caused by food backflow and reducing death caused by complications; and
5. Reinforcing personal swallow rehabilitation and improving living quality.

Accordingly, the present invention conforms to the legal requirements owing to its novelty, nonobviousness, and utility. However, the foregoing description is only embodiments of the present invention, not used to limit the scope and range of the present invention. Those equivalent changes or modifications made according to the shape, structure, feature, or spirit described in the claims of the present invention are included in the appended claims of the present invention.

1. A method for swallow safety monitoring, comprising steps of:
disposing one or more sound sensor at one or more chest location of a patient;
sensing a breath sound at said chest location of the patient in a non-diet period for generating a reference sound datum;
sensing a plurality of sensing sounds at said chest location of the patient in a diet period, and generating a plurality of sound sensing data; and
comparing said plurality of sound sensing data according to said reference sound datum and monitoring during said diet period, comparing said plurality of sound sensing data by using said reference sound datum as a threshold value, and generating an alarm message when a frequency or amplitude of one of said plurality of sound sensing data exceeds said threshold value.

2. The method for swallow safety monitoring of claim 1, wherein said step of sensing a plurality of sensing sounds at said chest location of the patient in a diet period further senses a plurality of postural horizontal angles of the patient for generating a plurality of sensing angle data.

3. The method for swallow safety monitoring of claim 1, further comprising a step of transmitting said reference sound data, said plurality of sound sensing data, and said diet period to an electronic device.

4. A device for swallow safety monitoring, comprising:
one or more sound sensor, disposed at one or more chest location of a patient, sensing a breath sound of the patient, and generating a reference sound datum;
a timer, synchronous with said sound sensor according to a diet period, triggering said sound sensor to sense a plurality of sensing sounds of the patient, and generate a plurality of sound sensing data; and
a monitoring unit, connected to said sound sensor and said timer, and monitoring according to said reference sound datum, said plurality of sound sensing data, and said diet period.

5. The device for swallow safety monitoring of claim 4, further comprising
an angle sensor, sensing the postural horizontal angle of the patient, and generating one or more reference angle datum; and
a storage unit, storing said reference sound datum, said plurality of sound sensing data, and said diet period.

6. The device for swallow safety monitoring of claim 5, further comprising a transmission module, connected to said storage unit for transmitting a first audio datum, a second audio datum, and said diet period to an electronic device via a wireless transmission interface.

7. The device for swallow safety monitoring of claim 5, further comprising a control unit, connected electrically to and controlling said monitoring unit, said sound sensor, said timer, and said angle sensor.

8. The device for swallow safety monitoring of claim 4, wherein said monitoring unit further comparing said plurality of sound sensing data by using said reference sound datum as a threshold value, and generating an alarm message when a frequency or amplitude of one of said plurality of sound sensing data exceeds said threshold value.

9. The device for swallow safety monitoring of claim 4, further comprising a display, connected to said sound sensor, said timer, and said angle sensor, and displaying.
